(19) United States
(12) Patent Application Publication
(10) Pub. No.: US 2012/0213798 A1
(43) Pub. Date: Aug. 23, 2012

(54) METHODS AND USES FOR TREATING HEPATIC ISCHEMIA AND REPERFUSION INJURY

(75) Inventors: Gary Levy, Thornhill (CA); Markus Selzner, Toronto (CA)

(73) Assignee: VERITAS THERAPEUTICS INC., Toronto, ON (CA)

(21) Appl. No.: 13/504,021

(22) PCT Filed: Oct. 27, 2010
(86) PCT No.: PCT/CA10/01689

§ 371 Date: Apr. 25, 2012

Related U.S. Application Data
(60) Provisional application No. 61/255,308, filed on Oct. 27, 2009.

(51) Int. Cl. A61K 39/395; G01N 33/53; A61P 41/00; A61P 1/16; A61P 35/00; C12Q 1/68; A61K 31/7088

(52) U.S. Cl. 424/158.1; 435/6.11; 435/7.1; 514/44 A

(57) ABSTRACT

The present disclosure provides a method of treating or preventing hepatic ischemia and reperfusion injury in an animal comprising administering an effective amount of an FGL2-FcγRIIB inhibitor to an animal in need thereof. Also provided herein are uses, compositions and screening assays for FGL2- FcγRIIB inhibitors useful in treating hepatic ischemia and reperfusion injury.

### CROSS REFERENCE TO RELATED APPLICATIONS

This application claims the benefit of priority of copending U.S. provisional application 61/255,308 filed on Oct. 27, 2009, the contents of which are incorporated herein by reference in their entirety.

### FIELD OF THE DISCLOSURE

The disclosure relates to methods and uses for treating hepatic ischemia and reperfusion injury. In particular, the disclosure relates to interfering with the interaction of FGL2 with Fc?RIIB to prevent or treat hepatic ischemia and reperfusion injury.

### BACKGROUND OF THE DISCLOSURE

Hepatic ischemia and reperfusion (I/Rp) injury is a common pathological process which occurs in the setting of major trauma, sepsis, and elective liver surgery including liver transplantation and can lead to severe hepatocellular dysfunction and organ failure. A critical event during reperfusion injury is sinusoidal endothelial cell (SEC) death, which occurs within minutes of reperfusion and precedes hepatocyte death by several hours (Natori 1999; Kohli 1999; Caldwell-Kenkel 1989; Ikeda 1992). Injury of the SEC after reperfusion causes microcirculatory disturbance associated with leukocyte and platelet adhesion and subsequent infiltration of inflammatory cells into the hepatic parenchyma (Clavien 1992; Vajdova 2004; Panes 1998; Lemasters 1997). It has been demonstrated that apoptosis of SEC occurs within minutes after reperfusion and protection of SEC apoptosis prevents hepatocyte death and liver injury (Kohli 1999; Natori 1999; Natori 2003). In contrast to the in vivo situation, the death of SEC in vitro occurs only after prolonged ischemia and 24 hr of re-oxygenation. This suggests that reperfusion of the whole organ in vivo provides mediators, which rapidly activates the apoptotic cascade in SEC.

FGL2 (fibrinogen like protein 2) is a 70 kDa glycoprotein that belongs to the fibrinogen-related superfamily of proteins which are involved in coagulation, cell adhesion, and transendothelial migration (Liu 2000; Sitrin 1998). FGL2 is expressed on the surface of different cell types, including endothelial cells (Ghanekar 2004), macrophages (Liu 2006), and T-cells (Marazzi 1998) and is constitutively expressed in many organs including liver, lung, kidneys, and heart (Ding 1997; Rychlik 2003). Previous work from the present inventors demonstrated that intra portal injection of both TNF-α and INFγ induces hepatocyte apoptosis in wild type mice but not in fgl2^{-/-} mice (Liu 2006). Since TNF-α and INFγ are both early key mediators of the ischemia/reperfusion cascade, this observation suggests the possibility that FGL2 might have an important role in the pathogenesis of hepatic I/Rp injury (Le Moine 2000; Lappas 2006).

The present inventors have recently identified the receptor for FGL2 and shown that FGL2 binding to the FcγRIIB receptor resulted in apoptosis of B-cells (Liu 2008). FcγReceptors are present on various cell populations including lymphocytes, macrophages, and SEC (Xu 2003; Mousavi 2007).

### SUMMARY OF THE DISCLOSURE

The present inventors have demonstrated that FGL2 is a critical mediator of sinusoidal endothelial cell (SEC) death during hepatic ischemia and reperfusion injury (I/Rp) through its interaction with FcγRIIB.

Accordingly, the present disclosure provides a method of treating or preventing hepatic ischemia and reperfusion injury (I/Rp) comprising administering an effective amount of an FGL2-FcγRIIB inhibitor to an animal in need thereof. The present disclosure also provides a use of an effective amount of an FGL2-FcγRIIB inhibitor for treating or preventing hepatic I/Rp in an animal in need thereof. Further provided is a use of an effective amount of an FGL2-FcγRIIB inhibitor in the preparation of a medicament for treating or preventing hepatic I/Rp in an animal in need thereof. Even further provided is an effective amount of an FGL2-FcγRIIB inhibitor for use in treating or preventing hepatic I/Rp in an animal in need thereof.

In one embodiment, the inhibitor comprises an FGL2 antibody or fragment thereof or an FcγRIIB antibody or fragment thereof. In another embodiment, the inhibitor comprises an antisense nucleic acid of a nucleic acid encoding FGL2. In yet another embodiment, the inhibitor comprises an antisense nucleic acid of a nucleic acid encoding FeγRIIB.

Also providing herein is a pharmaceutical composition for treating or preventing hepatic ischemia and reperfusion injury in an animal in need thereof comprising an FGL2-FcγRIIB inhibitor and a pharmaceutically acceptable carrier, diluent or excipient.

Further provided are screening assays for determining whether a test agent is an FGL2-FcγRIIB inhibitor useful for treating hepatic ischemia and reperfusion injury (I/Rp).

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The disclosure will now be described in relation to the drawings in which:

FIG. 1 shows serum FGL2 levels in sham operated animals, after 60 minutes ischemia alone, and after 60 minutes ischemia and various length of reperfusion (15 minutes, 30 minutes, 60 minutes, 90 minutes, 3 hr, 6 hr, 12 hr, and 24 hr). Serum FGL2 levels increase as early as 15 minutes post reperfusion with a peak at 30 minutes (*=p<0.001 compared to sham), FGL2 levels remain elevated in the serum up to 24 hr post reperfusion (n=5 at each time point).

FIG. 2 shows serum FGL2 levels in wild type mice and tnf-α^{-/-} mice following 60 minutes of ischemia and 30 minutes of reperfusion. FGL2 levels were significantly decreased in tnf-α^{-/-} mice in comparison to the wild type mice (p=0.02, student t-test) (n=5 each group).

FIG. 3 shows AST release following 60 minutes of ischemia and various lengths of reperfusion in wild type mice, fgl2^{-/-} mice, and mice pre-treated with a blocking anti-FGL2 antibody. After 3 hr and 6 hr of reperfusion the AST levels were significantly lower in fgl2^{-/-} mice and mice treated with a blocking anti-FGL2 antibody in comparison to the wild type (*p<0.01 fgl2^{-/-} and anti-FGL2 antibody treated mice compared with the Wild type). WT: wild type; FGL2-KO: FGL2 knockout mice (fgl2^{-/-}); FGL2 AB: anti-FGL2 antibody treated wild type mice; (n=5 each time point).

FIG. 4 shows A. liver necrosis following 60 minutes of ischemia and 24 hr of reperfusion. fgl2^{-/-} mice and anti-FGL2 antibody treated mice had significantly lower percentage of liver necrosis in comparison to the wild type. (*p<0.01 when compared to the wild type), (n=5 in each group). B. Trichrome staining of wild type mice (B-1) and FGL2 knockout mice (B-2) following 60 minutes of ischemia and 24 hr of reperfusion. Liver tissue of the wild type mice showed large areas of confluent necrosis compared to only a mild necrotic change in fgl2^{-/-} mice.

FIG. 5 shows animal survival after 75 minutes of total hepatic ischemia in wild type mice and fgl2^{-/-} mice. Deletion of fgl2 resulted in improved long-term survival following prolonged ischemic injury (80% vs 10% in wild type mice) (p=0.001, log rank) (n=10 in each group).

FIG. 6 shows A. cleaved caspase 3 positive cells following 60 minutes of ischemia and 90 minutes of reperfusion. Cleaved caspase 3 staining was present in the majority of SEC in wild type. In contrast, fgl2^{-/-} mice had a 5-times lower number of cleaved caspase 3 positive SEC in comparison to the wild type (p=0.001, students t-test) (n=5 in each group). B. Cleaved caspase 3 staining in wild type mice (B-1) and fgl2^{-/-} mice (B-2) following 1 hr ischemia and 90 minutes of reperfusion. Wild type mice have cleaved caspase 3 staining in majority of the SEC, while no cleaved caspase 3 staining is present in fgl2^{-/-} mice.

FIG. 7 shows A. immunohistochemistry of liver tissue for FcγRIIB/RIII receptor with a rat-anti-mouse FcγRIIB/RIII staining antibody. In wild type mice all SEC have positive staining for both receptors, while in FcγRIIB^{-/-} mice only macrophages and lymphocyte were stained positive. B. Flow cytometry of primary SEC isolated from wild type (FcγRIIB^{+/+}) and FcγRIIB knockout (FcγRIIB^{-/-}) mice. Only cells from wild type mice were positive for FcγRIIB/III antibody staining, while SEC derived from FcγRIIB^{-/-} were negative.

FIG. 8 shows staining for propidium iodide (PI) and pan-caspases (FAM-VAD-FMK) of isolated SEC challenged by recombinant FGL2. Exposure for 12 hr of SEC derived from wild type mice resulted in apoptosis of 30% of cells as shown by staining for propidium iodide and pan-caspase. In contrast, FGL2 administration did not induce apoptosis of cells derived from FcγRIIB^{-/-} mice.

FIG. 9 shows cleaved caspase 3 staining in wild type mice (A) and FcγRIIB^{-/-}(B) following 1 hr ischemia and 90 minutes of reperfusion. While wild type mice had cleaved caspase 3 staining in most SEC, no staining was observed in FcγRIIB^{-/-} mice.

### DETAILED DESCRIPTION OF THE DISCLOSURE

The present inventors have demonstrated that FGL2 is a critical mediator of sinusoidal endothelial cell (SEC) death during hepatic ischemia and reperfusion injury (I/Rp). In particular, the present inventors have determined that induction of FGL2 is downstream of ischemia/reperfusion (I/R)-induced TNF-α release and exerted its effects through ligation with the FcγRIIB receptor expressed on SEC, an event which results in SEC apoptosis and liver injury. Inhibition of FGL2-FcγRIIB binding not only prevented SEC apoptosis but decreased hepatocyte injury and markedly improved survival.

### Methods and Uses

The present disclosure provides a method of treating or preventing hepatic ischemia and reperfusion injury (I/Rp) comprising administering an effective amount of an FGL2-FcγRIIB inhibitor to an animal in need thereof. The present disclosure also provides a use of an effective amount of an FGL2-FcγRIIB inhibitor for treating or preventing hepatic I/Rp in an animal in need thereof. Further provided is a use of an effective amount of an FGL2-FcγRIIB inhibitor in the preparation of a medicament for treating or preventing hepatic I/Rp in an animal in need thereof. Even further provided is an effective amount of an FGL2-FcγRIIB inhibitor for use in treating or preventing hepatic I/Rp in an animal in need thereof.

The term “animal” includes all members of the animal kingdom, such as a mammal and optionally a human. Accordingly, in one embodiment, the animal is a mammal. In a particular embodiment, the animal is a human.

The phrase “hepatic ischemia and reperfusion injury” or “hepatic I/Rp” as used herein refers to the damage to liver tissue caused when blood supply returns to the tissue after a period of ischemia or lack of oxygen and nutrients. The damage includes, without limitation, sinusoidal endothelial cell

(SEC) death or apoptosis, which can be readily determined by a skilled person, for example, by detecting the presence of apoptotic markers, such as caspase-3 and propidium iodide.

In one embodiment, the animal in need of treatment or prevention of hepatic I/Rp has a major trauma, sepsis, liver injury or cancer.

Types of cancer in need of treatment or prevention of hepatic I/Rp include, without limitation, hepatocellular carcinoma (primary) and secondary tumors, such as breast and colon. In another embodiment, the animal in need of treatment or prevention of hepatic I/Rp has had or will have liver surgery, such as liver transplantation. In one embodiment, the FGL2-FcγRIIB inhibitor is used or administered prior to or in conjunction with the liver surgery to prevent the onset of hepatic ischemia and reperfusion injury.

As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing onset or spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term a “therapeutically effective amount”, “effective amount” or a “sufficient amount” of a compound or composition of the present disclosure is a quantity sufficient to, when administered to the subject, including a mammal, for example a human, effect beneficial or desired results, including clinical results, and, as such, an “effective amount” or synonym thereto depends upon the context in which it is being applied. For example, in the context of treating hepatic I/Rp, for example, it is an amount of the compound or composition sufficient to achieve such a treatment as compared to the response obtained without administration of the compound or composition. In the context of disease, therapeutically effective amounts of the compounds or compositions disclosed in the present disclosure are used to treat, modulate, attenuate, reverse, or affect hepatic I/Rp in an animal. An “effective amount” is intended to mean that amount of a compound or composition that is sufficient to treat, prevent or inhibit hepatic I/Rp. In some suitable embodiments, the amount of a given compound or composition will vary depending upon various factors, such as the given drug or compound, the pharmaceutical formulation, the route of administration, the type of disease or disorder, the identity of the subject or host being treated, and the like, but can nevertheless be routinely determined by one skilled in the art.

Also, as used herein, a “therapeutically effective amount” of a compound or composition of the present disclosure is an amount which prevents, inhibits, suppresses or reduces hepatic I/Rp in a subject as compared to a control. As defined herein, a therapeutically effective amount of a compound or composition of the present disclosure may be readily determined by one of ordinary skill by routine methods known in the art.

The term “FGL2 protein” or “fibroleukin protein ” or “fibrinogen-like 2 protein” as used herein includes FGL2 from any species or source and includes analogs and fragments or portions of FGL2 protein that can interact with FcγRIIB. The FGL2 protein may have any of the known published sequences for fgl2 which can be obtained from public sources such as GenBank. Examples of such sequences include, but are not limited to Accession Nos. AAL68855; P12804; Q14314; NP032039; AAG42269; AAD10825; AAB88815; AAB88814; NP006673; AAC16423; AAC16422; AAB92553. The fgl2 sequences can also be found in WO 98/51335 (published Nov. 19, 1998) and in Marazzi et al. (1998), Ruegg et al. (1995) and Yuwaraj et al. (2001)). The aforementioned sequences are incorporated herein by reference. The FGL2 protein can be obtained from any species, optionally a mammal including human and mouse.

The term “FcγRIIB” or “Fc gamma receptor II B”, also known as CD32, as used herein includes any FcγRIIB from any species or source and includes analogs and fragments or portions of FcγRIIB protein that can interact with FGL2. The FcγRIIB protein may have any of the known sequences for FcγRIIB which can be obtained from public sources such as GenBank. Examples of such sequences include but are not limited to Accession Nos. NM_{—}004001 and NM_{—}001077189. The aforementioned sequences are incorporated herein by reference. The FcγRIIB protein can be obtained from any species, optionally a mammal including human and mouse.

The term “FGL2-FcγRIIB inhibitor” as used herein includes any substance that is capable of inhibiting the interaction of FGL2 with the FcγRIIB receptor or is capable of inhibiting the expression or activity of FGL2 or FcγRIIB receptor. Substances having this property are identified readily using established in vitro and in vivo assays for measuring protein-protein interactions, for example, competitive binding assays, yeast two hybrid systems, coimmunoprecipitation, ELISA, Western Blot Analysis and Flow cytometry (FACS). Such substances include, without limitation, antisense nucleic acid molecules, proteins, antibodies (and fragments thereof), small molecule inhibitors and other substances. In one embodiment, the inhibitor is targeted to the liver. A person skilled in the art would readily understand how to target to the liver. In another embodiment, haemoglobin haptoglobin coupling is used to target to the liver (Levy et al. 2006).

Suitable antibodies include antibodies against FGL2 and/or FcγRIIB that block or inhibit the interaction between the two proteins.

The term “antibody” as used herein is intended to include monoclonal antibodies, polyclonal antibodies, chimeric and humanized antibodies. The antibody may be from recombinant sources and/or produced in transgenic animals. The term “antibody fragment” as used herein is intended to include without limitations Fab, Fab′, F(ab′)2, scFv, dsFv, ds-scFv, dimers, minibodies, diabodies, and multimers thereof, multispecific antibody fragments and domain antibodies. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques.

Antibodies to FGL2 are commercially available (H0001 0875-M01, Abnova). Antibodies to FcγRIIB are also commercially available (2.4G2, BD Pharmingen). However, a person skilled in the art will appreciate that one could produce other antibodies that interfere with the interaction of FGL2 and FcγRIIB. Suitable such antibodies include polyclonal antibodies, as well as monoclonal antibodies, and active fragments thereof. The antibodies can be non-human antibodies, but it is desirable to generate the more tolerated versions thereof such as chimeric and humanized antibodies. Accordingly, in one embodiment, the FGL2-FcγRIIB inhibitor is an FGL2 antibody or FcγRIIB antibody, such as an antibody to human FGL2 or human FcγRIIB.

The antibodies can be raised using, as antigen, the wild type FGL2 protein or FcγRIIB protein, or any fragment or domain thereof that retains the epitope(s) involved in their interaction. Antibodies so raised can then be screened using any of the protein-protein interaction assays noted above and established in the art, and antibodies that inhibit such interaction can be selected for use.

For example, by using either the wild type FGL2 or FcγRIIB, polyclonal antisera or monoclonal antibodies can be made using standard methods. A mammal, (e.g., a mouse, hamster, or rabbit) can be immunized with an immunogenic form of the peptide which elicits an antibody response in the mammal. The amino acid sequence for human FGL2, a useful immunogen for such purposes, is known from Levy et al. (US Pat. No. 6,403,089). Moreover, a technique for producing monoclonal anti-FGL2 antibody is described by Levy et al, in J. Biol. Chem., 1991, 286(3):1789 (see also Shalev et al. 2009, designated 6H12). The preparation of rabbit polyclonal antiserum against FGL2 is described in Ding et al. (1997) J. Virol. 71(12):9223. Techniques for conferring immunogenicity on a peptide include conjugation to carriers or other techniques well known in the art. For example, the protein or peptide can be administered in the presence of adjuvant. The progress of immunization can be monitored by detection of antibody titers in plasma or serum. Standard ELISA or other immunoassay procedures can be used with the immunogen as antigen to assess the levels of antibodies. Following immunization, antisera can be obtained and, if desired, polyclonal antibodies isolated from the sera.

To produce monoclonal antibodies, antibody-producing cells (lymphocytes) can be harvested from an immunized animal and fused with myeloma cells by standard somatic cell fusion procedures thus immortalizing these cells and yielding hybridoma cells. Such techniques are well known in the art, (e.g., the hybridoma technique originally developed by Kohler and Milstein (Nature 256, 495-497 (1975)) as well as other techniques such as the human B-cell hybridoma technique (Kozbor et al., lmmunol. Today 4, 72 (1983)), the EBV-hybridoma technique to produce human monoclonal antibodies (Cole et al. Monoclonal Antibodies in Cancer Therapy (1985) Allen R. Bliss, Inc., pages 77-96), and screening of combinatorial antibody libraries (Huse et al., Science 246, 1275 (1989)). Hybridoma cells can be screened immunochemically for production of antibodies specifically reactive with the peptide and the monoclonal antibodies can be isolated. Therefore, the disclosure also contemplates hybridoma cells secreting monoclonal antibodies with specificity for FGL2 or FcγRIIB.

Chimeric antibody derivatives, i.e., antibody molecules that combine a non-human animal variable region and a human constant region are also contemplated within the scope of the disclosure. Chimeric antibody molecules can include, for example, the antigen binding domain from an antibody of a mouse, rat, or other species, with human constant regions. Conventional methods may be used to make chimeric antibodies containing the immunoglobulin variable region which recognizes FGL2 or FcγRIIB (See, for example, Morrison et al., Proc. Natl Acad. Sci. U.S.A. 81,6851 (1985); Takeda et al., Nature 314, 452 (1985), Cabilly et al., U.S. Pat. No. 4,816,567; Boss et al., U.S. Pat. No. 4,816,397; Tanaguchi et al., European Patent Publication EP171496; European Patent Publication 0173494, United Kingdom patent GB 2177096B). It is expected that chimeric antibodies would be less immunogenic in a human subject than the corresponding non-chimeric antibody.

Monoclonal or chimeric antibodies specifically reactive with FGL2 or FcγRIIB can be further humanized by producing human constant region chimeras, in which parts of the variable regions, particularly the conserved framework regions of the antigen-binding domain, are of human origin and only the hypervariable regions are of non-human origin. Such immunoglobulin molecules may be made by techniques known in the art, (e.g., Teng et al., Proc. Natl. Acad. Sci. U.S.A., 80, 7308-7312 (1983); Kozbor et al., Immunology Today, 4, 7279 (1983); Olsson et al., Meth. Enzymol., 92, 3-16 (1982)), and PCT Publication W092/06193 or EP 0239400). Humanized antibodies can also be commercially produced (Scotgen Limited, 2 Holly Road, Twickenham, Middlesex, Great Britain.)

Specific antibodies, or antibody fragments, reactive against FGL2 or FcγRIIB may also be generated by screening expression libraries encoding immunoglobulin genes, or portions thereof, expressed in bacteria with peptides produced from the nucleic acid molecules of FGL2 or FcγRIIB. For example, complete Fab fragments, VH regions and FV regions can be expressed in bacteria using phage expression libraries (See for example Ward et al., Nature 341, 544-546: (1989); Huse et al., Science 246, 1275-1281 (1989); and McCafferty et al. Nature 348, 552-554 (1990)). Alternatively, a SCID-hu mouse, for example the model developed by Genpharm, can be used to produce antibodies or fragments thereof.

Thus, as noted above, FGL2-FcγRIIB inhibitors useful in the present disclosure include antibodies and antibody fragments that inhibit the interaction of FGL2 with FcγRIIB. Such antibodies can be raised against wild type FGL2 or FcγRIIB, e.g., human FGL2 or human FcγRIIB, or against fragments, domains or epitopes involved in mediating such interaction.

The FGL2-FcγRIIB inhibitors may also contain or be used to obtain or design “peptide mimetics”. For example, a peptide mimetic may be made to mimic the function of an FGL2-FcγRIIB inhibitor. “Peptide mimetics” are structures which serve as substitutes for peptides in interactions between molecules (See Morgan et al (1989), Ann. Reports Med. Chem. 24:243-252 for a review). Peptide mimetics include synthetic structures which may or may not contain amino acids and/or peptide bonds but retain the structural and functional features. Peptide mimetics also include molecules incorporating peptides into larger molecules with other functional elements (e.g., as described in WO 99/25044). Peptide mimetics also include peptoids, oligopeptoids (Simon et al (1972) Proc. Natl. Acad, Sci USA 89:9367) and peptide libraries containing peptides of a designed length representing all possible sequences of amino acids corresponding to an FGL2-FcγRIIB inhibitor peptide.

Peptide mimetics may be designed based on information obtained by systematic replacement of L-amino acids by D-amino acids, replacement of side chains with groups having different electronic properties, and by systematic replacement of peptide bonds with amide bond replacements. Local conformational constraints can also be introduced to determine conformational requirements for activity of a candidate peptide mimetic. The mimetics may include isosteric amide bonds, or D-amino acids to stabilize or promote reverse turn conformations and to help stabilize the molecule. Cyclic amino acid analogues may be used to constrain amino acid residues to particular conformational states. The mimetics can also include mimics of the secondary structures of the proteins described herein. These structures can model the 3-dimensional orientation of amino acid residues into the known secondary conformations of proteins. Peptoids may also be used which are oligomers of N-substituted amino acids and can be used as motifs for the generation of chemically diverse libraries of novel molecules.

It will further be appreciated that agents useful to inhibit the interaction of FGL2 with FcγRIIB are not limited to antibodies or peptide mimetics that interfere with the interaction of FGL2 with FcγRIIB, but instead include agents that inactivate genetic expression of FGL2 or FcγRIIB such as sense and anti-sense RNA and DNA constructs that bind genomic DNA or mRNA encoding FGL2 or FcγRIIB that compete with FGL2 for binding to FcγRIIB. The disclosure also includes small interfering RNA (siRNA) that target and inhibit the expression of FGL2 or FcγRIIB.

Accordingly, the present disclosure includes the use of antisense oligonucleotides to inhibit the activity of FGL2 or FcγRIIB. In one embodiment, the FGL2-FcγRIIB inhibitor is an antisense nucleic acid sequence of an FGL2 encoding sequence. In another embodiment, the FGL2-FcγRIIB inhibitor is an antisense nucleic acid sequence of an FcγRIIB encoding sequence.

The term “antisense oligonucleotide” as used herein means a nucleotide sequence that is complementary to its target. The term “oligonucleotide” refers to an oligomer or polymer of nucleotide or nucleoside monomers consisting of naturally occurring bases, sugars, and intersugar (backbone) linkages. The term also includes modified or substituted oligomers comprising non-naturally occurring monomers or portions thereof, which function similarly. Such modified or substituted oligonucleotides may be preferred over naturally occurring forms because of properties such as enhanced cellular uptake, or increased stability in the presence of nucleases. The term also includes chimeric oligonucleotides which contain two or more chemically distinct regions. For example, chimeric oligonucleotides may contain at least one region of modified nucleotides that confer beneficial properties (e.g. increased nuclease resistance, increased uptake into cells), or two or more oligonucleotides of the disclosure may be joined to form a chimeric oligonucleotide.

The antisense oligonucleotides of the present disclosure may be ribonucleic or deoxyribonucleic acids and may contain naturally occurring bases including adenine, guanine, cytosine, thymidine and uracil. The oligonucleotides may also contain modified bases such as xanthine, hypoxanthine, 2-aminoadenine, 6-methyl, 2-propyl and other alkyl adenines, 5-halo uracil, 5-halo cytosine, 6-aza uracil, 6-aza cytosine and 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thiolalkyl guanines, 8-hydroxyl guanine and other 8-substituted guanines, other aza and deaza uracils, thymidines, cytosines, adenines, or guanines, 5-trifluoromethyl uracil and 5-trifluoro cytosine.

Other antisense oligonucleotides of the disclosure may contain modified phosphorous, oxygen heteroatoms in the phosphate backbone, short chain alkyl or cycloalkyl intersugar linkages or short chain heteroatomic or heterocyclic intersugar linkages. For example, the antisense oligonucleotides may contain phosphorothioates, phosphotriesters, methyl phosphonates, and phosphorodithioates. In an embodiment, there are phosphorothioate bonds links between the four to six 3′-terminus bases. In another embodiment phosphorothioate bonds link all the nucleotides.

The antisense oligonucleotides of the disclosure may also comprise nucleotide analogs that may be better suited as therapeutic or experimental reagents. An example of an oligonucleotide analogue is a peptide nucleic acid (PNA) wherein the deoxyribose (or ribose) phosphate backbone in the DNA (or RNA), is replaced with a polyamide backbone which is similar to that found in peptides (RE. Nielsen, et al. Science 1991, 254, 1497). PNA analogues have been shown to be resistant to degradation by enzymes and to have extended lives in vivo and in vitro. PNAs also bind stronger to a complementary DNA sequence due to the lack of charge repulsion between the PNA strand and the DNA strand. Other oligonucleotides may contain nucleotides containing polymer backbones, cyclic backbones, or acyclic backbones. For example, the nucleotides may have morpholino backbone structures (U.S. Pat. No. 5,034,506). Oligonucleotides may also contain groups such as reporter groups, a group for improving the pharmacokinetic properties of an oligonucleotide, or a group for improving the pharmacodynamic properties of an antisense oligonucleotide. Antisense oligonucleotides may also have sugar mimetics.

The antisense nucleic acid molecules may be constructed using chemical synthesis and enzymatic ligation reactions using procedures known in the art. The antisense nucleic acid molecules of the disclosure or a fragment thereof, may be chemically synthesized using naturally occurring nucleotides or variously modified nucleotides designed to increase the biological stability of the molecules or to increase the physical stability of the duplex formed with mRNA or the native gene e.g. phosphorothioate derivatives and acridine substituted nucleotides. The antisense sequences may be produced biologically using an expression vector introduced into cells in the form of a recombinant plasmid, phagemid or attenuated virus in which antisense sequences are produced under the control of a high efficiency regulatory region, the activity of which may be determined by the cell type into which the vector is introduced.

The antisense oligonucleotides may be introduced into tissues or cells using techniques in the art including vectors (retroviral vectors, adenoviral vectors and DNA virus vectors) or physical techniques such as microinjection. The antisense oligonucleotides may be directly administered in vivo or may be used to transfect cells in vitro which are then administered in vivo.

The present disclosure also includes the use of siRNA to inhibit the activity of FGL2 or FcγRIIB.

The term “siRNA” refers to a short inhibitory RNA that can be used to silence gene expression of a specific gene. The siRNA can be a short RNA hairpin (e.g. shRNA) that activates a cellular degradation pathway directed at mRNAs corresponding to the siRNA. Methods of designing specific siRNA molecules and administering them are known to a person skilled in the art. It is known in the art that efficient silencing is obtained with siRNA duplex complexes paired to have a two nucleotide 3′ overhang. Adding two thymidine nucleotides is thought to add nuclease resistance. A person skilled in the art will recognize that other nucleotides can also be added.

Also included herein are aptamers to FGL2 or FcγRIIB. Aptamers are short strands of nucleic acids that can adopt highly specific 3-dimensional conformations. Aptamers can exhibit high binding affinity and specificity to a target molecule. These properties allow such molecules to specifically inhibit the functional activity of proteins. Thus, in another embodiment, the FGL2-FcγRIIB inhibitor is an aptamer that binds and inhibits the interaction of FGL2 with FcγRIIB.

The nucleic acid molecules disclosed herein may be incorporated in a known manner into an appropriate expression vector which ensures good expression of the polypeptides. Various constructs can be used to deliver nucleic acid molecules described herein. For example retroviral constructs such as lentiviral constructs are useful for expressing physiological levels of protein. Possible expression vectors include but are not limited to cosmids, plasmids, or modified viruses (e.g. replication defective retroviruses, adenoviruses and adeno-associated viruses), so long as the vector is compatible with the host cell used. The expression vectors are “suitable for transformation of a host cell”, which means that the expression vectors contain a nucleic acid molecule and regulatory sequences selected on the basis of the host cells to be used for expression, which is operatively linked to the nucleic acid molecule. Operatively linked is intended to mean that the nucleic acid is linked to regulatory sequences in a manner which allows expression of the nucleic acid.

The disclosure therefore includes a recombinant expression vector containing a nucleic acid molecule disclosed herein, or a fragment thereof, and the necessary regulatory sequences for the transcription and translation of the inserted protein-sequence.

Suitable regulatory sequences may be derived from a variety of sources, including bacterial, fungal, viral, mammalian, or insect genes (For example, see the regulatory sequences described in Goeddel, Gene Expression Technology: Methods in Enzymology 185, Academic Press, San Diego, Calif. (1990)). Selection of appropriate regulatory sequences is dependent on the host cell chosen as discussed below, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector.

The recombinant expression vectors may also contain a selectable marker gene which facilitates the selection of host cells transformed or transfected with a recombinant molecule disclosed herein. Examples of selectable marker genes are genes encoding a protein such as G418 and hygromycin which confer resistance to certain drugs, β-galactosidase, chloramphenicol acetyltransferase, firefly luciferase, or an immunoglobulin or portion thereof such as the Fc portion of an immunoglobulin optionally IgG. Transcription of the selectable marker gene is monitored by changes in the concentration of the selectable marker protein such as β-galactosidase, chloramphenicol acetyltransferase, or firefly luciferase. If the selectable marker gene encodes a protein conferring antibiotic resistance such as neomycin resistance transformant cells can be selected with G418. Cells that have incorporated the selectable marker gene will survive, while the other cells die. This makes it possible to visualize and assay for expression of the recombinant expression vectors disclosed herein and in particular to determine the effect of a mutation on expression and phenotype. It will be appreciated that selectable markers can be introduced on a separate vector from the nucleic acid of interest.

Suitable host cells include a wide variety of prokaryotic and eukaryotic host cells. For example, the proteins of the disclosure may be expressed in bacterial cells such as E. coli, insect cells (using baculovirus), yeast cells or mammalian cells. Other suitable host cells can be found in Goeddel (Goeddel, Gene Expression Technology: Methods in Enzymology 185, Academic Press, San Diego, Calif. 1990).

The FGL2-FcγRIIB inhibitors may be used alone or in combination with other known agents useful for treating or preventing hepatic I/Rp. The currently used agents for treating hepatic I/Rp include, without limitation, Pentoxyfyline, glycine, arginine, steroids and anti-TNF.

When used in combination with other agents useful in treating hepatic I/Rp, the inhibitors are suitably administered contemporaneously with those agents. As used herein, “contemporaneous administration” of two substances to an individual means providing each of the two substances so that they are both biologically active in the individual at the same time. The exact details of the administration will depend on the pharmacokinetics of the two substances in the presence of each other, and can include administering the two substances within a few hours of each other, or even administering one substance within 24 hours of administration of the other, if the pharmacokinetics are suitable. Design of suitable dosing regimens is routine for one skilled in the art. In particular embodiments, two substances will be administered substantially simultaneously, i.e., within minutes of each other, or in a single composition that contains both substances.

### Pharmaceutical Compositions

The disclosure also provides a pharmaceutical composition for preventing or treating hepatic I/Rp in an animal in need thereof comprising an FGL2-FcγRIIB inhibitor and a pharmaceutically acceptable carrier, diluent or excipient.

The term “pharmaceutically acceptable” as used herein means compatible with the treatment of animals, such as humans.

The FGL2-FcγRIIB inhibitors may be formulated into pharmaceutical compositions for administration to subjects in a biologically compatible form suitable for administration in vivo. By “biologically compatible form suitable for administration in vivo” is meant a form of the substance to be administered in which any toxic effects are outweighed by the therapeutic effects. The substances may be administered to living organisms including humans, and animals. Administration of a therapeutically active amount of the pharmaceutical compositions of the present disclosure is defined as an amount effective, at dosages and for periods of time necessary to achieve the desired result. For example, a therapeutically active amount of a substance may vary according to factors such as the disease state, age, sex, and weight of the individual, and the ability of protein to elicit a desired response in the individual. Dosage regime may be adjusted to provide the optimum therapeutic response. For example, several divided doses may be administered daily or the dose may be proportionally reduced as indicated by the exigencies of the therapeutic situation.

The active substance may be administered in a convenient manner such as by injection (subcutaneous, intravenous, intramuscular, etc.), oral administration, inhalation, intranasal, transdermal administration (such as topical cream or ointment, etc.), or suppository applications. In one embodiment, the active substance is administered by inhalation or intranasally. Depending on the route of administration, the active substance may be coated in a material to protect the compound from the action of enzymes, acids and other natural conditions which may inactivate the compound. The active substance may be formulated into delayed release formulations such that mucus overproduction can be prevented for longer periods of time than a conventional formulation.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences (2000—20th edition) Mack Publishing Company). On this basis, the compositions include, albeit not exclusively, solutions of the substances in association with one or more pharmaceutically acceptable vehicles or diluents, and contained in buffered solutions with a suitable pH and iso-osmotic with the physiological fluids.

### Screening Assays

The disclosure provides a screening assay for determining whether a test agent is an FGL2-FcγRIIB inhibitor useful for treating hepatic I/Rp, said assay comprising
(a) determining binding of FGL2 with FcγRIIB in the presence of a test agent; and
(b) comparing the binding in (a) with the binding in the absence of the test agent;
wherein a lower amount of binding in the presence of the test agent indicates that the test agent is useful in treating hepatic I/Rp.

The phrase “determining the binding of FGL2 with FcγRIIB” as used herein refers to any method of determining protein-protein binding and includes, without limitation, techniques known in the art. For example, protein-protein binding can be determined in vitro by methods such as competitive binding assays and/or in vivo by methods such as a yeast two hybrid system or FACS.

In another embodiment, the disclosure provides a screening assay for determining whether a test agent is an FGL2-FcγRIIB inhibitor useful for treating hepatic I/Rp, said assay comprising
(a) providing SEC cells;
(b) contacting the SEC cells with FGL2 in the presence of a test agent;
(c) detecting SEC-FGL2 binding; and
(d) comparing binding in step (c) with a control in the absence of the test agent;
wherein a decrease in binding in the presence of test agent indicates that the test agent is useful for treating hepatic I/Rp.

In one embodiment, the binding in step (c) is detected by flow cytometry.

In yet another embodiment, the disclosure provides a screening assay for determining whether a test agent is an FGL2-FcγRIIB inhibitor useful for treating hepatic I/Rp, said assay comprising:
(a) providing SEC cells;
(b) incubating or contacting SEC of step (a) with FGL2, such as recombinant FGL2, in the presence of a test agent; and
(c) determining SEC apoptosis compared to a control in the absence of SEC apoptosis;
wherein a decrease in apoptosis in the presence of the test agent indicates that the test agent is useful for treating hepatic I/Rp.

Techniques for determining SEC apoptosis are known in the art and include measuring apoptotic markers such as caspase-3 or propidium iodide.

The test agent can be any compound which one wishes to test including, but not limited to, proteins, peptides, nucleic acids (including RNA, DNA, antisense oligonucleotide, peptide nucleic acids), carbohydrates, organic compounds, small molecules, natural products, library extracts, bodily fluids and other samples that one wishes to test for inhibitory activity.

The above disclosure generally describes the present disclosure. A more complete understanding can be obtained by reference to the following specific examples. These examples are described solely for the purpose of illustration and are not intended to limit the scope of the disclosure. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Materials and Methods

### Animals

All experiments were performed in male C57/BL6 mice 6 weeks of age. The animals received a standard laboratory diet with water and food ad libitum. Mice were kept under constant environmental conditions with a 12 hr light-dark cycle (light 7:00 a.m. to 7:00 p.m.). All surgical procedures were performed under aseptic conditions between 7:00 a.m. and 11 a.m. to avoid circadian variations. Euthanasia was performed using exsanguination. Animals received humane care in compliance with the Toronto General Research Institute Animal Care Committee.

### FGL2 and FcγRIIB Knockout Mice

Production of fgl2^{-/-} mice has been described previously (Marsden 2003; Mendicino 2005). Mice of fgl2^{-/-} genotype were backcrossed to C57BL/6 for 10 generations. The LacZ reporter and PGK-neogene were inserted within the first of two coding exons of the fgl2 gene enabled detection of fgl2 transcription by γ-galactosidase (γ-gal) expression.

FcγRIIB knockout mice on a C57BL/6 background were purchased from Jackson laboratories (Bar Habor, Mass., USA).

### Anti FGL2 Antibody Treatment

Blocking anti-FGL2 antibodies (H0001 0875-M01) were purchased from Abnova (Walnut, Calif., USA). 50 μg of the antibody were injected iv 30 minutes prior to the beginning of ischemia.

### Partial Hepatic Ischemia

A model of segmental (70%) hepatic ischemia was used. Mice were anesthetized with inhalation of isoflurane (Pittman-Moore, Chicago, Ill.). After a midline laparotomy, the structures to the left of the porta hepatis (hepatic artery, portal vein and bile duct), serving the left and median liver lobes, were occluded for 60 minutes. This method of partial hepatic ischemia prevents mesenteric venous congestion by permitting portal decompression through the right and caudate lobes (Yadav, 1998). Reperfusion was initiated by removal of the clamp. Animals were again anesthesized at different periods after reperfusion and liver biopsies taken for further evaluation.

### Total Hepatic Ischemia

Total hepatic ischemia was produced by placing a microvascular clamp distally of the branches to the right and caudate hepatic lobes for 75 minutes as previously described (Selzner 2006). Ischemia was induced in the middle and left hepatic lobes while ensuring of portal vein drainage by the non-ischemic liver tissue. Prior to the removal of the clamp, the non-ischemic lobes were resected and reperfusion was performed with the left and middle lobe in situ.

### Parameters of Hepatocyte Injury

The degree of hepatic injury was assessed by measurement of serum aspartate aminotransferase (AST), an established marker of hepatocellular injury in the rodent liver (Selzner 2002). AST serum levels were analyzed using a serum multiple analyzer (Johnson & Johnson®, Ektachem DTSC II multianalyzer).

### TUNEL Staining

At various times after ischemia/reperfusion, mice were euthanized and their livers were perfused with freshly prepared 4% paraformaldehyde in PBS (pH 7.2) under a constant pressure of 10 cm H₂O for 5 min through the portal vein. Each liver was cut in 3 to 5 mm sections that were stored in 70% alcohol after additional overnight fixation in 4% paraformaldehyde. Tissues were then incubated in 30% sucrose/PBS, embedded in 7.5% gelatin and finally frozen in isopentane submerged in dry ice and 95% alcohol slush. Sections of 5 μm were placed on silanized slides, and were treated with terminal deoxynucleotidyl transferase from calf thymus in the presence of fluorescein-dUTP and d-NTP, according to the supplier's recommended protocol (Boehringer Mannheim, Ind., IN catalog #1767305). This was followed by post-staining using horeseradish peroxidase conjugated anti-fluorescein antibody, and then developed using diaminobenzidine/H₂O₂. Positive and negative controls were performed using test sections pretreated with DNAse I and staining without deoxynucleotide substrate, respectively. Thirty randomly-selected fields were investigated per slide by a blinded investigator (MS) to determine the percentage of TUNEL positive cells.

### Cleaved Caspase 3 Immunohistochemistry

As an alternate measure of apoptosis, cleaved caspase 3 in-situ end-labeling technique on formalin-fixed, paraffin-embedded material was performed as previously described (Wijsman 1993). Briefly, 4 microns sections were dewaxed in xylene followed by graded alcohol treament. The sections were treated with 1% pepsin (Sigma: P7125) in 0.01N HCl at pH 2.0. Endogenous peroxidase and endogenous biotin activities were blocked using 3% aqueous hydrogen peroxide and avidin/biotin blocking kit (Vector Labs. Inc.). Following buffer treatment (buffer contains 50 mM TRIS-HCl (pH 7.5); 50 mM MgCl2.6H20; 100 mM p-Mercaptoethanol; and 0.005% BSA), the sections were incubated with Biotin-nucleotide cocktail in water bath at 37° C. for 1 to 1.5 hours, and washed in PBS. Ultra Streptavidin Horseradish Peroxidase Labeling Reagent (ID Labs inc. cat.# BP2378) was then applied for 30 min at room temperature, and washed in PBS, and developed with freshly prepared Nova Red solution (Vector Labs. Inc.). After washing in running water, Mayer's hematoxylin counterstaining was applied, sectioned, coverslipped, and reviewed using Leica light microscope DM4500B.

### TNF-α Levels in Plasma

Whole blood (0.5 mL) obtained by vena cava puncture was centrifuged at 4,000 g for 15 minutes at 4° C. to obtain plasma, and 50 μL aliquots were snap frozen for TNF-α assay. Plasma TNF-α levels were determined by an enzyme-linked immunosorbent assay using a commercial kit (Quantikine Murine Tumor Necrosis Factor-a Assay; R & D, Minn., MN).

### FGL2 ELISA

FGL2 levels in mouse serum were quantified as previously described (Shalev 2009). Briefly, plates were coated and incubated overnight with 2 μg/ml monoclonal anti-FGL2 (6H12) (IgG1) as a capture antibody. Plasma samples (50 μl) were added to each well, and following a 1 hour incubation at 37° C. and 3 washes with TTBS, the wells were incubated with 2 μg/ml polyclonal rabbit anti-FGL2 antibody for 2 hr at 37° C. The plate was washed again and polyclonal anti-FGL2 binding was detected with secondary HRP-conjugated anti-rabbit antibody. Tetrametylbenzidine (TMB) was then added and absorbance was measured at 450 nm using an ELISA plate reader (Shalev 2009).

### Determination of Hepatic Necrosis

Histology was evaluated by a blinded investigator (AO).

Hepatocellular necrosis was determined in H&E stained tissue sections by a point counting method using a semi-quantitative scale as previously described (Kohli 1999). Thirty random fields were investigated per slide to determine the area of necrosis. In this study, only grade 3 injury with destruction of hepatic cords was counted as necrosis.

### Isolation of Sinusoidal Endothelial Cells

Sinusoidal endothelial cells were isolated as previously described (McQueen 1987). Briefly, the infrahepatic vena cava was cannulated with a 22 g angiocath. After occlusion of the suprahepatic vena cava with a microclamp and transsection of the portal vein the liver was retrograde flushed with solution 1 containing EGTA in Hank's Balanced Salt Solution without calcium and magnesium, followed by perfusion with solution 2 containing collagenase IV (100 units/ml), 50 μg/ml gentamicin, 10 mM Hepes in Hanks balance solution with calcium and magnesium. After perfusion the liver was excised and minced with a scalpel. The homogenate was filtered and then centrifuged at 50 g for 7 minutes. The supernatant was centrifuged again at 360 g for 10 minutes. The pellet was resuspended in Willliams E medium+10% fetal bovine serum and layered over 16% metrizamide (Accurate Chemical, estbury, N.Y.) and centrifuged at 2300 g for 45 minutes at 4° C. The supernatant was then incubated with CD146 microbeads (Miltenyi Biotec, Aburn, US) and passed through a LS MACS (Magnetic cell separation) column. After removal of the magnetic field the column was washed and they were collected. Viability was confirmed above 95% as determined with trypan blue exclusion. Purity was above 97% as evaluated with Flow cytometry using an anti CD31 antibody.

### Statistical Analysis

The data were analyzed with the SPSS 11.5 statistic package (Chicago, Ill.). A Student's t-test was used for the comparison of continuous variables, while a Chi-square test was applied for categorical outcome. A log rank analysis was performed for survival experiments. The results are presented as mean±SD and were considered significant at the level of p<0.05.

### Results

### FGL2 is Released Immediately Following Hepatic I/Rp

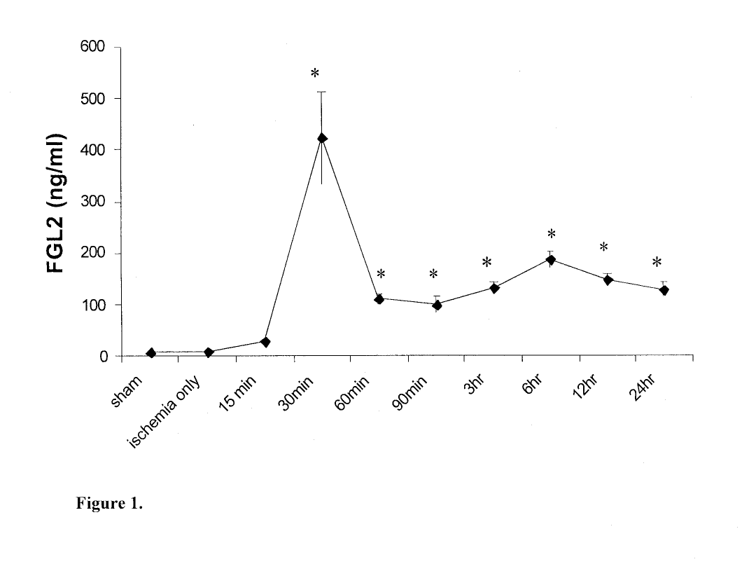

The plasma levels of FGL2 were determined in mice after sham operation (laparotomy) and after 60 minutes of ischemia with various length of reperfusion (15, 30, 60 minutes, 3 hr, 6 hr, 12 hr, and 24 hr). Plasma FGL2 levels were low in mice with either sham laparotomy or ischemia alone (7±1 ng/ml and 9±1 ng/ml). In contrast, 60 minutes of ischemia and 30 minutes of reperfusion resulted in a 70-fold increase of plasma levels of FGL2 (423±90 ng/ml, p<0.0001 vs sham). Plasma FGL2 levels remained elevated up to 24 hr following reperfusion (FIG. 1).

### FGL2 Release Following Hepatic I/Rp is TNF-α Dependent

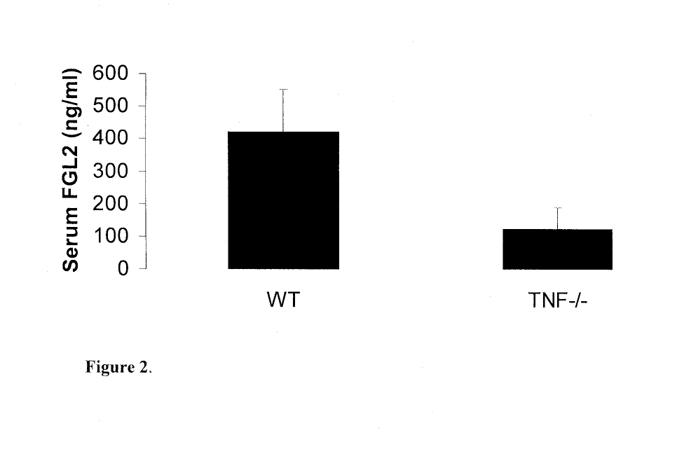

To determine whether FGL2 release after reperfusion injury is downstream of TNF-α release, plasma levels of FGL2 were compared between wild type and tnf-α^{-/-} mice following 60 minutes of ischemia and 30 minutes of reperfusion. As shown in FIG. 2, levels of FGL2 were significantly reduced in tnf-α^{-/-} mice in comparison to wild type mice (123±64 ng/ml vs 423±90 ng/ml, p<0.02). In contrast to the findings above, following 60 minutes ischemia and 30 minutes of reperfusion. fgl2^{-/-} mice and wild type mice had identical plasma levels of TNF-α (10.2+2.5 pg/mg vs 13.5±3.6 pg/mg, p=0.2). Therefore, these results show that following liver I/Rp, FGL2 is downstream of TNF-α release.

### FGL2 is a Critical Mediator of Hepatic I/Rp

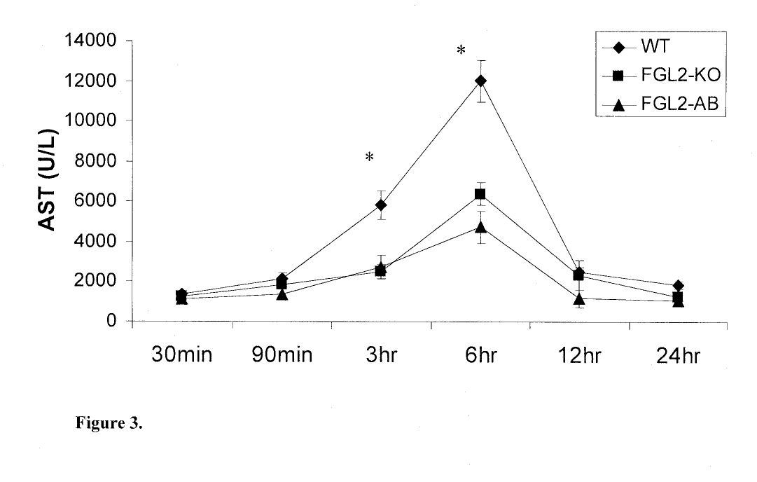

To determine the role of FGL2 in reperfusion injury, two approaches were used. FGL2 activity was blocked by either targeted deletion of fgl2 (fgl2^{-/-} mice), or inhibition of FGL2 by blocking antibody (H0001 0875-M01, Abnova, Walnut, Calif.). FIG. 3 shows a time course of AST release following each of these approaches in comparison to I/Rp in wild type animals. At 3 and 6 hours of reperfusion, both of the FGL2 inhibitory approaches attenuated liver injury, as evidenced by a 50% reduction in AST levels.

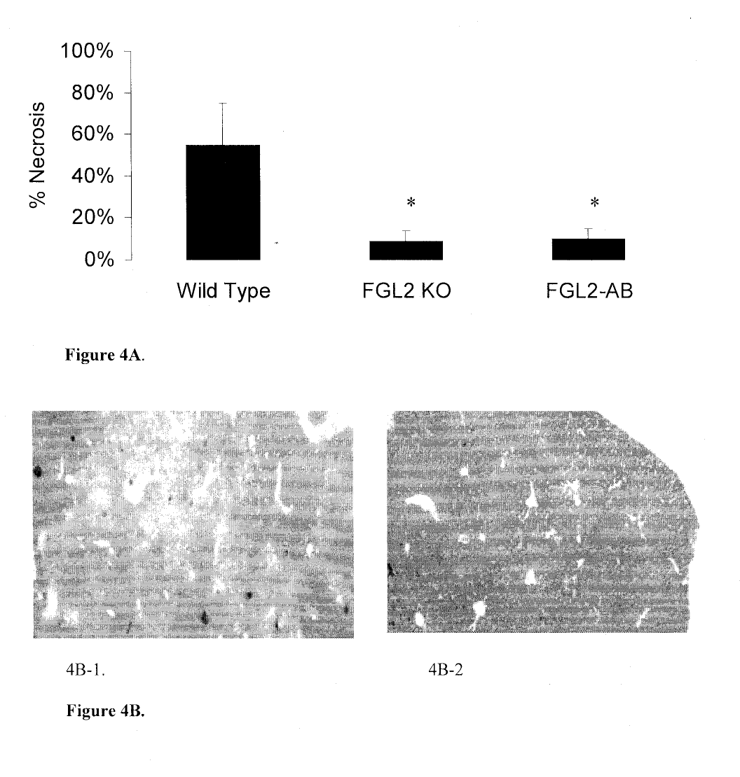

Hepatocellular necrosis after 24 hr of reperfusion was chosen as a second endpoint to evaluate the severity of ischemic injury. Following 60 minutes of ischemia and 24 hr of reperfusion, light microscopic examination of liver necrosis revealed large areas of confluent necrosis in wild type animals, while in fgl2^{-/-} animals, necrosis was largely absent (FIG. 4A). This effect is shown in FIG. 4B where 55±20% of the hepatocytes were necrotic in wild type mice whereas only 9±5% and 10±5% of necrosis was observed in both fgl2^{-/-} and anti-FGL2 antibody treated mice, respectively.

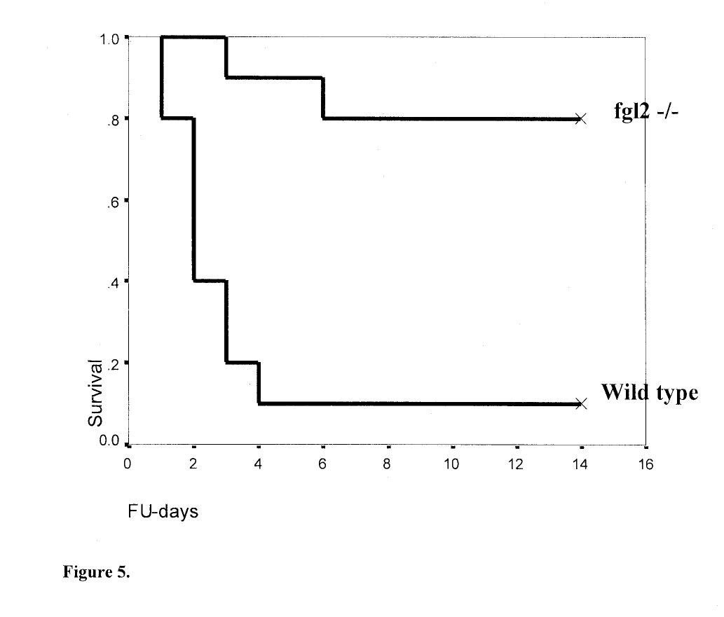

Finally, the impact of targeted fgl2 deletion on animal survival was determined after a prolonged period of ischemia. Following 75 minutes of total hepatic ischemia, there was a 90% mortality (9 out of 10) in wild type mice within three days, while 80% (8 out of 10) of fgl2^{-/-} mice survived the 14 day experimental period (p<0.001) (FIG. 5).

### FGL2 Induces SEC Apoptosis Following Hepatic I/Rp Injury

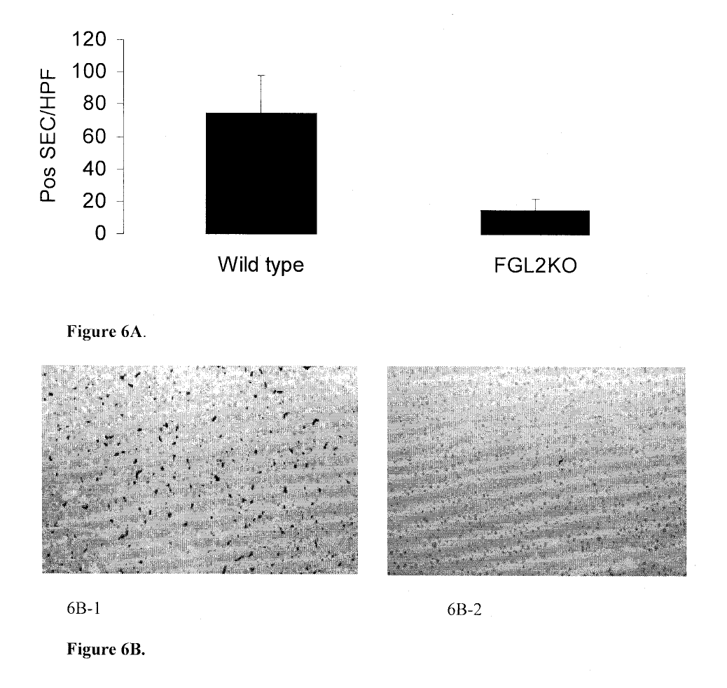

SEC apoptosis is known to contribute to hepatocellular injury following I/Rp. To assess whether FGL2 induced SEC and hepatocyte apoptosis following reperfusion injury, two markers of apoptosis were measured: cleaved caspase 3 and TUNEL staining. Immunohistochemistry for cleaved caspase 3 was performed after 60 minutes of ischemia and 90 minutes of reperfusion. 82±15% of the SEC in the wild type mice were positive for cleaved caspase 3, while only 10±7% of the SEC in the fgl2^{-/-} mice stained for cleaved caspase 3 (p<0.02) (FIG. 6A & B). By contrast, hepatocytes were negative for cleaved caspase 3 staining at this early time point of reperfusion in both wild type as well as in the fgl2^{-/-} mice.

TUNEL staining was used as a late marker of apoptosis. 60 minute of ischemia and 90 minutes of reperfusion induced TUNEL staining of 65±12% SEC in wild type mice, whereas only 11±4% of SEC were TUNEL positive in fgl2^{-/-} mice (p=0.02).

### Hepatic SEC Express FcγRIIB but not FcγRIII Receptor

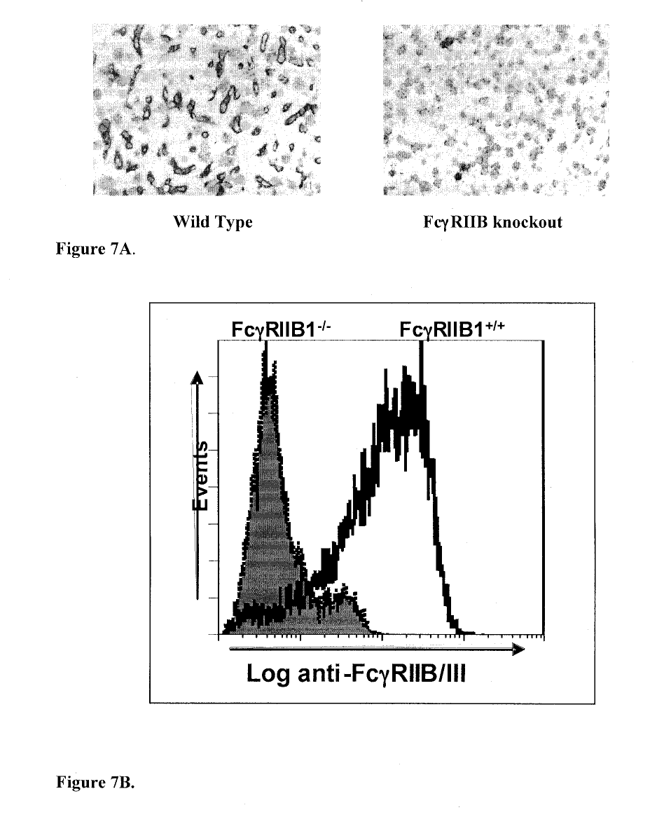

To address the role of FcγR in liver injury, expression in liver tissue was first examined. Immunohistochemistry of liver tissue from wild type mice using a rat-anti-mouse FcγRIIB/RIII antibody (2.4G2) (BD Pharmingen, Mississauga, Calif.) demonstrated strong staining of all SEC lining and Kupffer cells in wild type mice. In contrast, FcγRIIB^{-/-} mice had no staining on SEC, while Kupffer cells and lymphocytes were stained (FIG. 7A). This finding suggested that FcγRII was expressed on SEC, while FcγRIII was not. In a second approach, primary SEC were isolated from wild type mice and FcγRIIB^{-/-}. Sinusoidal endothelial cells were stained again with the rat-anti-mouse FcγRIIB/RIII antibody (2.4G2) (BD Pharmingen, Mississauga, Calif.), which binds to both FcγRIIB and FcγRIII receptors. Flow cytometry showed strong staining in the SEC from wild type mice, but not FcγRIIB^{-/-} mice (FIG. 7B).

FGL2 Interacts with the FcγRIIB Receptor and Induces SEC Apoptosis

Having demonstrated the presence of FcγRII receptors on SEC, studies were performed to determine that role in I/Rp injury. In B-lymphocytes, FGL2 was shown to bind to FcγRII receptors and mediate apoptotic cell death. In the first set of experiments, it was determined whether FGL2 binds to the FcγRIIB receptor on SEC. SEC were freshly isolated from wild type and FcγRIIB^{-/-} mice and were incubated with biotin labeled FGL2 and counter stained with a secondary antibody (SA-727C, BD Pharmingen, Mississauga, Calif.). SEC-FGL2 binding was detected by flow cytometry. Labeled FGL2 binding was detected on SEC from wild type mice, but not from FcγRIIB^{-/-}

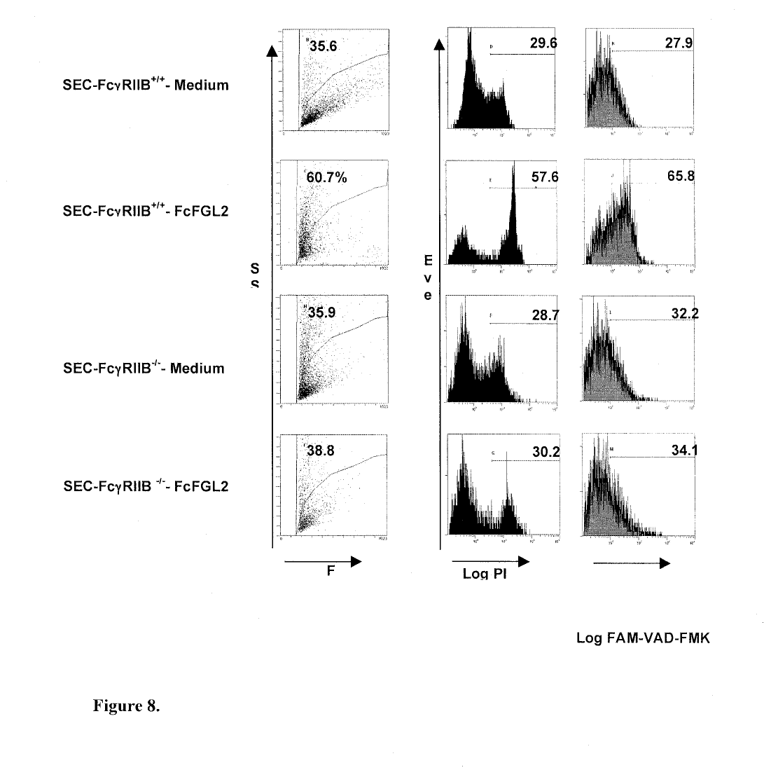

Second, it was investigated whether FGL2-FcγRII binding induces SEC death. Isolated SEC from wild type mice and FcγRIIB^{-/-} mice were incubated in cell culture with recombinant FGL2 (500 nM) for 12 hr or with serum as a control. SEC apoptosis was determined by flow cytometry determining pan-caspase activation by FAM-VAD-FMK staining, while propidium iodide staining was used as a second endpoint for cell death. After 12 hr incubation with FGL2, 66%±7% of wild type SEC cells stained positive for FAM-VAD-FMK, while 30%±5% of wild type SEC after exposure to medium alone (p<0.001). The FGL2-induced increase in FAM-VAD-FMK staining SEC was not observed in cells obtained from FcγRIIB knockout mice (32%±4%) and did not differ from medium-treated cells in knockout animals (34%±3%) (p=0.9). Similarly, propidium iodide staining was higher in wild type SEC after FGL2 exposure (58%±8%) compared to wild type cells treated with medium alone (30%±4%), or to FGL2-treated SEC from FcγRIIB^{-/-} mice (FIG. 8).

Deletion of the FcγRIIB Receptor Prevents SEC Apoptosis Following Hepatic I/Rp in vivo

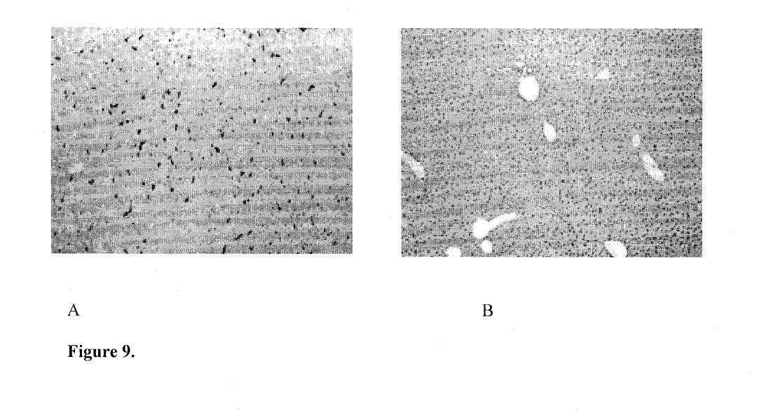

SEC apoptosis was investigated in wild type mice, FcγRIIB^{-/-} mice and wild type mice treated with a blocking anti-FcγRII antibody. Apoptosis was determined by staining for cleaved caspase 3 and TUNEL. Following 60 minutes of ischemia and 90 minutes of reperfusion 80±17% of the SEC in the wild type mice were positive for cleaved caspase 3 staining, compared to only 5±3% of the SEC in FcγRIIB−/− mice (p=0.001) (FIG. 9).

### Discussion

The present inventors and others have demonstrated that following ischemic injury SEC undergo a caspase dependent apoptotic cell death within minutes of reperfusion, while hepatocyte death occurs only several hours later (Natori 2003; Natori 1999; Kohli 1999; Gao 1998; Sindram 2000; Sindram 2001). In contrast, in vitro SEC are much more resistant to hypoxia and re-oxygenation and develop cell death only after several hours of hypoxia and prolonged re-oxygenation (Samarasinghe 2000; Samarasinghe 1996; Zeilinger 1997). Samarasinghe et al. compared in vitro the susceptibility of different liver cell types to hypoxia and found that SEC were even more resistant to prolonged hypoxic injury than hepatocytes or Kupffer cell (Samarasinghe 1996). In their study only 10% of SEC underwent cell death despite 24 hr hypoxia (PO₂<1 mmHg), and even after 8 hr hypoxia and 24 hr of re-oxygenation only 40% of the SEC expressed features of necrotic cell death. The differences in SEC susceptibility against ischemia and re-oxygenation in vivo and in vitro indicate that the rapid death of SEC in vivo is probably due to the release of specific mediators at the time of reperfusion. The results of the present disclosure indicate that one of the mediators inducing rapid SEC death in vivo is FGL2.

FGL2 is a multipotent protein which has been associated with inflammatory response in different settings such as rejection and viral hepatitis (Han 2008; Shalev 2009; Su 2008; Wilczynski 2006). In the present model the plasma levels of FGL2 remained low after ischemia alone. In contrast, as early as 30 minutes of reperfusion a 70-fold increase in FGL2 levels was observed indicating that FGL2 is released shortly after reperfusion of ischemic liver. The FGL2 release was dependent on the presence of TNF-α a key mediator of reperfusion injury. TNF-α release by Kupffer cells or lymphocytes occurs rapidly after reperfusion and is present within minutes (Rüdiger 2000; Le Moine 2000; Selzner 2008). Conversely, TNF-α levels were not affected by FGL2 deletion, demonstrating that FGL2 is located downstream to TNF-α in the signaling cascade of reperfusion injury. TNF-α has been linked to different pathways within the reperfusion injury cascade. This includes direct activation of TNFR1 on hepatocytes with subsequent activation of apoptotic cascades which ultimately results in cell death (Rüdiger 2000). In addition, TNF-α induces neutrophils adhesion and infiltration following ischemic injury (Kokura 2000; Schwabe 2006). The present disclosure describes a novel role for TNF-α following I/Rp, which is the induction of FGL2 release and therefore targeting SEC death.

Liu et al (Liu 2008) demonstrated that FGL2 binds to FcγRIIB and FcγRIII receptors on B lymphocytes, macrophages and dendritic cells. Fcγ-receptors have been traditionally associated with regulation of the immune response with either activation or suppression of antigen presenting cells or lymphocytes. Activation of the FcγRIII receptor is associated with a cell activation signal (Pearse 1999), while inhibitory responses have been described after activation of FcγRIIB. Furthermore binding of FGL2 to the FcγRIIB receptor on B lymphocytes induced activation of caspase 3 and apoptotic cell death. In the present model, SEC only expressed the inhibitory FcγRIIB receptors but not FcγRIII receptors, which might explain the strong induction of SEC apoptosis in response to FGL2 in vitro and in vivo following I/R in a FGL2-dependent manner. The cell of origin of FGL2 following liver I/R and the mechanism whereby it interacts with SEC to cause apoptosis is an area of ongoing investigation.

The important role of the FcγRIIB receptor for the induction of liver injury has been reported previously by Xu et al (Xu 2003) in a model of anti-Fas antibody (Jo2) mediated liver injury. Jo2 injection resulted in complete destruction of the SEC in wild type mice but not FcγRIIB^{-/-} mice. The authors proposed that Jo2 induced hepatocyte injury was the result of binding to the Fas receptor with activation of the co-localized FcγRIIB receptor. Although Fas does not seem to play a role in hepatic reperfusion injury (Rudiger 2002), it is possible that activation of the FcγRIIB receptor on SEC can be induced by binding of different mediators, including Fas and FGL2.

Several potential mechanisms could explain the effect of FGL2-FcγRIIB binding after reperfusion on the induction of liver injury. Hepatocyte injury is dependent on sinusoidal adhesion of platelets and leukocytes, and subsequent infiltration of liver parenchyma by neutrophils and CD4+ cells resulting in the generation of oxygen free radicals and induction of oxidative stress (Sindram 2002; Jaeschke 1999; Jaeschke 1991; Ramaiah 2007). One potential explanation that links SEC FGL2-FcγRIIB binding to subsequent hepatocyte injury is disruption of the sinusoidal linage conferred by SEC. This interruption might facilitate attachment and/or penetration of infiltrating cells within liver tissue. In the present study protection of the SEC by either targeting strategies against FGL2 or FcγRIIB resulted in a strong protection against hepatocyte injury and improved animal survival even after a prolonged ischemic period. Alternatively, FGL2 binding to FcγRIIB receptor could induce up-regulation of adhesion molecules resulting in adhesion of platelets or leukocytes, which could provide additional death signals to the SEC.

In summary, FGL2-FcγRIIB interactions represent a novel pathway of SEC death following hepatic ischemia and reperfusion. Rapid caspase dependent apoptosis of the SEC precedes hepatocyte death and likely promotes hepatic reperfusion injury.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### References:

1. Natori, S., M. Selzner, K. Valentino, L. Fritz, A. Srinivasan, P.-A. Clavien, and G. Gores. 1999. Apoptosis of sinusoidal endothelial cells occurs during liver preservation injury by a caspase-dependent mechanism. Transplantation 68:89-96.

2. Kohli, V., M. Selzner, J. Madden, R. Bentley, and P. Clavien. 1999. Endothelial cell and hepatocyte deaths occur by apoptosis after ischemia-reperfusion injury in the rat liver. Translantation 67:1099-1105.

3. Caldwell-Kenkel, J. R. Currin, Y. Tanaka, R. Thurman, and J. Lemasters. 1989. Reperfusion injury to endothelial cells following cold ischemic storage of rat livers. Hepatology 10:292-299.

4. Ikeda, T., K. Yanaga, K. Kishikawa, S. Kakizoe, M. Shimada, and K. Sugimachi. 1992. Ischemic injury in liver transplantation: difference in injury sites between warm and cold ischemia in rats. Hepatology 16:454-461.

5. Clavien, P. A., P. R. Harvey, and S. M. Strasberg. 1992. Preservation and reperfusion injuries in liver allografts. An overview and synthesis of current studies. Transplantation 53:957-978.

6. Vajdova, K., S. Heinrich, Y. Tian, R. Graf, and P. A. Clavien. 2004. Ischemic preconditioning and intermittent clamping improve murine hepatic microcirculation and Kupffer cell function after ischemic injury. Liver Transpl 10:520-528.

7. Panes, J., and D. N. Granger. 1998. Leukocyte-endothelial cell interactions: molecular mechanisms and implications in gastrointestinal disease. Gastroenterology 114:1066-1090.

8. Lemasters, J. J., and R. G. Thurman. 1997. Reperfusion injury after liver preservation for transplantation. Annu Rev Pharmacol Toxicol 37:327-338.

9. Natori, S., H. Higuchi, P. Contreras, and G. J. Gores. 2003. The caspase inhibitor IDN-6556 prevents caspase activation and apoptosis in sinusoidal endothelial cells during liver preservation injury. Liver Transpl 9:278-284.

10. Liu, X., and T. H. Piela-Smith. 2000. Fibrin(ogen)-induced expression of ICAM-1 and chemokines in human synovial fibroblasts. J Immunol 165:5255-5261.

11. Sitrin, R. G., P. M. Pan, S. Srikanth, and R. F. Todd, 3rd. 1998. Fibrinogen activates NF-kappa B transcription factors in mononuclear phagocytes. J Immunol 161:1462-1470.

12. Ghanekar, A., M. Mendicino, H. Liu, W. He, M. Liu, R. Zhong, M. J. Phillips, G. A. Levy, and D. R. Grant. 2004. Endothelial induction of fgl2 contributes to thrombosis during acute vascular xenograft rejection. Journal of Immunology 172:5693-5701.

13. Liu, M., M. Mendicino, Q. Ning, A. Ghanekar, W. He, I. McGilvray, I. Shalev, D. Pivato, D. A. Clark, M. J. Phillips, and G. A. Levy. 2006. Cytokine-induced hepatic apoptosis is dependent on FGL2/fibroleukin: the role of Sp1/Sp3 and STAT1/PU.1 composite cis elements. J Immunol 176:7028-7038.

14. Marazzi, S., S. Blum, R. Hartmann, D. Gundersen, M. Schreyer, S. Argraves, V. von Fliedner, R. Pytela, and C. Ruegg. 1998. Characterization of human fibroleukin, a fibrinogen-like protein secreted by T lymphocytes. J Immunol 161:138-147.

15. Ding, J. W., Q. Ning, M. F. Liu, A. Lai, J. Leibowitz, K. M. Peltekian, E. H. Cole, L.S. Fung, C. Holloway, P. A. Marsden, H. Yeger, M. J. Phillips, and G. A. Levy. 1997. Fulminant hepatic failure in murine hepatitis virus strain 3 infection: tissue-specific expression of a novel fgl2 prothrombinase.[erratum appears in J Virol 1998 Apr;72(4):3504]. Journal of Virology 71:9223-9230.

16. Rychlik, D. F., E. K. Chien, D. Wolff, S. Phillippe, and M. Phillippe. 2003. Cloning and tissue expression of the tissue prothrombinase FgI-2 in the Sprague-Dawley rat. J Soc Gynecol Investig 10:67-73.

17. Le Moine, O., H. Louis, A. Demols, F. Desalle, F. Demoor, E. Quertinmont, M. Goldman, and J. Deviere. 2000. Cold liver ischemia-reperfusion injury critically depends on liver T cells and is improved by donor pretreatment with interleukin 10 in mice. Hepatology 31:1266-1274.

18. Lappas, C. M., Y. J. Day, M. A. Marshall, V. H. Engelhard, and J. Linden. 2006. Adenosine A2A receptor activation reduces hepatic ischemia reperfusion injury by inhibiting CD1d-dependent NKT cell activation. J Exp Med 203:2639-2648.

19. Liu, H., I. Shalev, J. Manuel, W. He, E. Leung, J. Crookshank, M. F. Liu, J. Diao, M. Cattral, D. A. Clark, D. E. Isenman, R. M. Gorczynski, D. R. Grant, L. Zhang, M. J. Phillips, M. I. Cybulsky, and G. A. Levy. 2008. The FGL2-FcgammaRllB pathway: a novel mechanism leading to immunosuppression. Eur J Immunol 38:3114-3126.

20. Xu, Y., A. J. Szalai, T. Zhou, K. R. Zinn, T. R. Chaudhuri, X. Li, W. J. Koopman, and R. P. Kimberly. 2003. Fc gamma Rs modulate cytotoxicity of anti-Fas antibodies: implications for agonistic antibody-based therapeutics. J Immunol 171:562-568.

21. Mousavi, S. A., M. Sporstol, C. Fladeby, R. Kjeken, N. Barois, and T. Berg. 2007. Receptor-mediated endocytosis of immune complexes in rat liver sinusoidal endothelial cells is mediated by FcgammaRIIb2. Hepatology 46:871-884.

22. Gao, W., R. C. Bentley, J. F. Madden, and P. A. Clavien. 1998. Apoptosis of sinusoidal endothelial cells is a critical mechanism of prevention injury in rat liver transplantation. Hepatology 27:1652-1660.

23. Sindram, D., R. J. Porte, M. R. Hoffman, R. C. Bentley, and P. A. Clavien. 2000. Platelets induce sinusoidal endothelial cell apoptosis upon reperfusion of the cold ischemic rat liver. Gastroenterology 118:183-191

24. Sindram, D., R. Prote, M. Hoffmann, R. Bentley, and P. Clavien. 2001. Synergism between platelets and leukocytes in inducing endothelial cell apoptosis in the cold ischemic rat liver: a Kupffer-mediated injury. FASEB J 15:1230-1232.

25. Samarasinghe, D., M. Tapner, and G. Farrell. 2000. Role of oxidative stress in hypoxia-reoxygenation injury to cultured rat hepatic sinusoidal endothelial cells. Hepatology 31:160-165.

26. Samarasinghe, D. A., and G. C. Farrell. 1996. The central role of sinusoidal endothelial cells in hepatic hypoxia-reoxygenation injury in the rat. Hepatology 24:1230-1237.

27. Zeilinger, K., B. Tonnelier, A. Tschesche, W. Nehls, G. Spatkowski, R. Schindler, E. Riedel, Y. C. Gerlach, and P. Neuhaus. 1997. Effect of energy substrates on the preservation outcome of hepatocytes and sinusoidal endothelial cells in an experimental hypoxia/reoxygenation model. Transplant Proc 29:403-405.

28. Han, M., W. Yan, W. Guo, D. Xi, Y. Zhou, W. Li, S. Gao, M. Liu, G. Levy, X. Luo, and Q. Ning. 2008. Hepatitis B virus-induced hFGL2 transcription is dependent on c-Ets-2 and MAPK signal pathway. J Biol Chem 283:32715-32729.

29. Shalev, I., K. M. Wong, K. Foerster, Y. Zhu, C. Chan, A. Maknojia, J. Zhang, X. Z. Ma, X. C. Yang, J. F. Gao, H. Liu, N. Selzner, D. A. Clark, O. Adeyi, M. J. Phillips, R. R. Gorczynski, D. Grant, I. McGilvray, and G. Levy. 2009. The novel CD4+CD25+ regulatory T cell effector molecule fibrinogen-like protein 2 contributes to the outcome of murine fulminant viral hepatitis. Hepatology 49:387-397.

30. Su, K., F. Chen, W. M. Yan, Q. L. Zeng, L. Xu, D. Xi, B. Pi, X. P. Luo, and Q. Ning. 2008. Fibrinogen-like protein 2/fibroleukin prothrombinase contributes to tumor hypercoagulability via IL-2 and IFN-gamma. World J Gastroenterol 14:5980-5989.

31. Wilczynski, J. R. 2006. Immunological analogy between allograft rejection, recurrent abortion and pre-eclampsia—the same basic mechanism? Hum Immunol 67:492-511.

32. Rüdiger, H., and P. Clavien. 2000. A new role for TNF-alpha in mediating hepatocye apoptosis in the normothermic ischemic liver. Hepatology 32:708A.

33. Selzner, M., N. Selzner, L. Chen, I. Borozan, J. Sun, M. Xue-Zhong, J. Zhang, and I. McGilvray. 2008. Exaggerated up-regulation of TNF-dependent apoptosis in the older mouse liver following reperfusion injury: Targeting liver protective strategies to donor age.submitted.

34. Kokura, S., R. E. Wolf, T. Yoshikawa, D. N. Granger, and T. Y. Aw. 2000. T-lymphocyte-derived tumor necrosis factor exacerbates anoxia-reoxygenation-induced neutrophil-endothelial cell adhesion. Circulation Research 86:205-213.

35. Schwabe, R. F., and D. A. Brenner. 2006. Mechanisms of Liver Injury. I. TNF-alpha-induced liver injury: role of IKK, JNK, and ROS pathways. Am J Physiol Gastrointest Liver Physiol 290:G583-589.

36. Pearse, R. N., T. Kawabe, S. Bolland, R. Guinamard, T. Kurosaki, and J. V. Ravetch. 1999. SHIP recruitment attenuates Fc gamma RIIB-induced B cell apoptosis. Immunity 10:753-760.

37. Rudiger, H., and P. Clavien. 2002. Tumor necrosis factor-alpha, but not Fas, mediates hepatocellular apoptosis in the murine ischemic liver. Gastroenterology 122:202-210.

38. Sindram, D., H. A. Rüdiger, A. G. Upadhya, S. M. Strasberg, and P. A. Clavien. 2002. Ischemic preconditioning protects against cold ischemic injury through an oxidative stress dependent mechanism. J Hepatol 36:78-84.

39. Jaeschke, H. 1999. Kupffer cell-induced oxidant stress during hepatic ischemia-reperfusion: Does the controversy continue? Hepatology 30:1527-1528.

40. Jaeschke, H., and A. Farhood. 1991. Neutrophil and Kupffer cell-induced oxidant stess and ischemia-reperfusion injury in rat liver in vivo. Am J Physiol 260:G355-362.

41. Ramaiah, S. K., and H. Jaeschke. 2007. Role of neutrophils in the pathogenesis of acute inflammatory liver injury. Toxicol Pathol 35:757-766.

42. Marsden, P. A., Q. Ning, L. S. Fung, X. Luo, Y. Chen, M. Mendicino, A. Ghanekar, J. A. Scott, T. Miller, C. W. Chan, M. W. Chan, W. He, R. M. Gorczynski, D. R. Grant, D. A. Clark, M. J. Phillips, and G. A. Levy. 2003. The Fgl2/fibroleukin prothrombinase contributes to immunologically mediated thrombosis in experimental and human viral hepatitis. J Clin Invest 112:58-66.

43. Mendicino, M., M. Liu, A. Ghanekar, W. He, C. Koscik, I. Shalev, M. Javadi, J. Turnbull, W. Chen, L. Fung, S. Sakamoto, P. Marsden, T. K. Waddell, M. J. Phillips, R. Gorczynski, G. A. Levy, and D. Grant. 2005. Targeted deletion of Fgl-2/fibroleukin in the donor modulates immunologic response and acute vascular rejection in cardiac xenografts. Circulation 112:248-256.

44. Yadav, S. S., W. Gao, R. C. Harland, and P. A. Clavien. 1998. A new and simple technique of total hepatic ischemia in the mouse. Transplantation 65:1433-1436.

45. Selzner, N., M. Selzner, W. Jochum, B. Amann-Vesti, R. Graf, and P. A. Clavien. 2006. Mouse livers with macrosteatosis are more susceptible to normothermic ischemic injury than those with microsteatosis. J Hepatol 44:694-701.

46. Selzner, M., H. Rudiger, N. Malekkiani, D. Thomas, D. Sindram, and P. Clavien. 2002. Transgenic mice overexpressing Bcl-2 are resistent to hepatic ischemia and reperfusion. J Hepatol 36:218-225.

47. Wijsman, J. H., R. R. Jonker, R. Keijzer, C. J. van de Velde, C. J. Cornelisse, and J. H. van Dierendonck. 1993. A new method to detect apoptosis in paraffin sections: in situ end-labeling of fragmented DNA. J Histochem Cytochem 41:7-12.

48. Kohli, V., J. F. Madden, R. C. Bentley, and P. A. Clavien. 1999. Calpain mediates ischemic injury of the liver through modulation of apoptosis and necrosis. Gastroenterology 116:168-178.

49. McQueen, C. A., and G. M. Williams. 1987. The hepatocyte primary culture/DNA repair test using hepatocytes from several species. Cell Biol Toxicol 3:209-218.

50. Levy et al. 2006. Targeted delivery of ribavirin improves outcome of murine viral fulminant hepatitis via enhanced anti viral activity Hepatology 43:581.

1. A method of treating or preventing hepatic ischemia and reperfusion injury (I/Rp) comprising administering an effective amount of an FGL2-FcγRIIB inhibitor to an animal in need thereof.

2. The method of claim 1, wherein the animal has a major trauma, sepsis liver injury or cancer.

3. The method of claim 1, wherein the animal has had or will have liver surgery.

4. The method of claim 3, wherein the liver surgery is liver transplantation.

5. The method of claim 1, wherein the inhibitor comprises an FGL2 antibody or fragment thereof.

6. The method of claim 1, wherein the inhibitor comprises an antisense nucleic acid of a nucleic acid encoding FGL2.

7. The method of claim 1, wherein the inhibitor comprises an FcγRIIB antibody or fragment thereof.

8. The method of claim 1, wherein the inhibitor comprises an antisense nucleic acid of a nucleic acid encoding FcγRIIB.

9. The method of claim 1, wherein the animal is a human.

10. The method of claim 1, further comprising administering Pentoxyfyline, glycine, arginine, steroids or anti-TNF.

11. A pharmaceutical composition for treating or preventing hepatic ischemia and reperfusion injury in an animal in need thereof comprising an FGL2-FcγRIIB inhibitor and a pharmaceutically acceptable carrier, diluent or excipient.

12. The pharmaceutical composition of claim 11, wherein the inhibitor is an antibody against FGL2.

13. The pharmaceutical composition of claim 11, wherein the inhibitor is an antibody against FcγRIIB.

14. A screening assay for determining whether a test agent is an FGL2-FcγRIIB inhibitor useful for treating hepatic ischemia and reperfusion injury (I/Rp), the assay comprising:
a) detecting binding of FGL2 with FcγRIIB in the presence of a test agent; and
b) comparing the binding in step (a) with the binding in the absence of the test agent;
wherein a lower amount of binding in the presence of the test agent indicates that the test agent is useful in treating hepatic I/Rp.

15. A screening assay for determining whether a test agent is an FGL2-FcγRIIB inhibitor useful for treating hepatic I/Rp, said assay comprising
(a) providing SEC cells;
(b) contacting the SEC cells with FGL2 in the presence of a test agent;
(c) detecting SEC-FGL2 binding or SEC apoptosis; and
(d) comparing binding or apoptosis in step (c) with a control in the absence of the test agent;
wherein a decrease in binding or apoptosis in the presence of test agent indicates that the test agent is useful for treating hepatic I/Rp.

16. The screening assay of claim 15, wherein the binding in step (c) is detected by flow cytometry.

17. (canceled)
